SEQUENCE LISTING (4 sequences)

SEQ ID NO: 1 - protein, 31 aa, Homo sapiens
HAEGTFTSDVSSYLEGQAAKEFIAWLVKGRG

SEQ ID NO: 2 - protein, 31 aa, synthetic construct
HGEGTFTSDVSSYLEEQAAKEFIAWLVKGGG

SEQ ID NO: 3 - protein, 5 aa, synthetic construct
GGGGS

SEQ ID NO: 4 - protein, 275 aa, synthetic construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2026/0365712 A1
(43) Pub. Date: Nov. 19, 2026

(54) METHODS OF PRODUCING FC-CONTAINING PROTEINS

(71) Applicant: Eli Lilly and Company, Indianapolis, IN (US)

(72) Inventors: Ciara Dunne, Cork (IE); Brian Gerard Kearns, Scotch Plains, NJ (US); Ashley Lynn Rettberg, Hillsborough, NJ (US)

(73) Assignee: Eli Lilly and Company, Indianapolis, IN (US)

(21) Appl. No.: 19/691,511

(22) Filed: May 28, 2026

Related U.S. Application Data
(63) Continuation of application No. PCT/US2024/058820, filed on Dec. 6, 2024.
(60) Provisional application No. 63/607,593, filed on Dec. 8, 2023.

(51) Int. Cl. C07K 14/605; C12N 5/00; C12N 5/071

(52) U.S. Cl. CPC C07K 14/605; C12N 5/0018; C12N 5/0682; C12N 2500/32

(57) ABSTRACT

Provided herein are improved methods for producing an Fc-containing protein. The methods generally involve culturing a cell that expresses the Fc-containing protein in a production bioreactor for a first period of time followed by supplying a first nutrient feed and culturing the cell for a second period of time followed by supplying a second nutrient feed. The methods may further involve culturing the cell for a third period of time, followed by supplying a third nutrient feed.

### RELATED APPLICATIONS

This application claims the benefit under 35 U.S.C. § 365 (c) and § 120 and is a continuation of International Patent Application Number PCT/US2024/058820, filed Dec. 6, 2024, which claims the benefit under 35 U.S.C. § 119 (e) of U.S. Patent Application Ser. No. 63/607,593, filed Dec. 8, 2023, the entire contents of these applications are incorporated by reference herein.

### REFERENCE TO AN ELECTRONIC SEQUENCE LISTING

The contents of the electronic sequence listing (E072870018US02-SEQ-KZM.xml; Size: 4,812 bytes; and Date of Creation: May 4, 2026) are herein incorporated by reference in their entirety.

### BACKGROUND

Production of recombinant Fc-containing proteins for therapeutic use typically involves expression of the proteins in cultured cells. Cell culture conditions can affect the yield and/or quality of Fc-containing proteins produced by the cultured cells. In particular, sub-optimal cell culture conditions can result in an increase in the amount of protease clipping and/or an increase in unfavorable glycosylation of Fc-containing proteins produced.

Accordingly, there is a need for improved cell culture methods for producing recombinant Fc-containing proteins that minimize issues such as protease clipping and/or generating undesired glycosylation while maximizing protein yield and quality.

### SUMMARY

The present disclosure provides improved methods for producing Fc-containing proteins. These methods are particularly advantageous in that they can reduce protease clipping and unfavorable glycosylation of Fc-containing proteins. As demonstrated in the Examples herein, these methods result in efficient, high titer production of Fc-containing proteins with desirable quality profiles. The methods generally involve culturing a cell (e.g., a mammalian cell) that expresses the Fc-containing protein in a production bioreactor for a first period of time followed by supplying a first nutrient feed and culturing the cell for a second period of time followed by supplying a second nutrient feed. In some embodiments, the methods further involve culturing the cell for a third period of time, followed by supplying a third nutrient feed.

Accordingly, in one aspect, the instant disclosure provides a method for producing dulaglutide, the method comprising culturing a mammalian cell that expresses the dulaglutide in a production bioreactor for a total culture time, wherein a first nutrient feed is supplied to the production bioreactor after a first portion of the total culture time; a second nutrient feed is supplied to the production bioreactor after a second portion of the total culture time; and a third nutrient feed is supplied to the production bioreactor after a third portion of the total culture time, such that the dulaglutide is produced by the mammalian cell.

In an embodiment, the first nutrient feed, the second nutrient feed, and/or the third nutrient feed comprises L-tyrosine. In an embodiment, each of the first nutrient feed, the second nutrient feed, and the third nutrient feed comprises L-tyrosine.

In an embodiment, the total culture time is 12-16 days. In an embodiment, the total culture time is about 14 days. In an embodiment, the first portion of the total culture time is 2-4 days after initiation of the culturing. In an embodiment, the first nutrient feed is supplied about 3 days after initiation of the culturing. In an embodiment, the second portion of the total culture time is 5-7 days after initiation of the culturing. In an embodiment, the second nutrient feed is supplied about 6 days after initiation of the culturing. In an embodiment, the third portion of the total culture time is 8-10 days after initiation of the culturing. In an embodiment, the third nutrient feed is supplied about 9 days after initiation of the culturing. In an embodiment, the first nutrient feed is supplied about 3 days after initiation of the culturing, the second nutrient feed is supplied about 6 days after initiation of the culturing, and the third nutrient feed is supplied about 9 days after initiation of the culturing.

In an embodiment, the maximum viable cell density (VCD) in the production bioreactor is between 8×10⁶ and 14×10⁶ cells per milliliter.

In an embodiment, the mammalian cell is selected from the group consisting of a COS cell, a CHO cell, a BHK cell, an MDCK cell, a HEK293 cell, a HEK293T cell, a HeLa cell, an NS0 cell, a PER.C6 cell, a VERO cell, a CRL7O3O cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. In an embodiment, the mammalian cell is a CHO cell.

In an embodiment, less than about 4% of the dulaglutide produced is an N-terminally clipped variant. In an embodiment, the N-terminally clipped variant is missing an N-terminal histidine residue or an N-terminal histidine residue and glycine residue. In an embodiment, between about 1.5% and 2.6% of the dulaglutide produced comprises a G2F glycan. In an embodiment, between about 10.6% and 15.4% of the dulaglutide produced comprises a GIF glycan. In an embodiment, between about 74.7% and 80% of the dulaglutide produced comprises a GOF glycan. In an embodiment, not more than about 1% of the dulaglutide produced comprises a Man-5 glycan. In an embodiment, 1.5-2.6% of the dulaglutide produced comprises a G2F glycan, 10.6-15.4% of the dulaglutide produced comprises a GIF glycan, 74.7-80% of the dulaglutide produced comprises a GOF glycan and not more than about 1% of the dulaglutide produced comprises a Man-5 glycan.

In another aspect, provided herein is dulaglutide produced by any of the methods described herein.

### BRIEF DESCRIPTION OF DRAWINGS

FIG. 1 shows that an updated dulaglutide manufacturing scheme, according to aspects of the present disclosure, generates an increased dulaglutide titer at day 14 relative to an initial dulaglutide manufacturing scheme. Mammalian cell cultures in two test tanks were supplied with a 1.5× concentration nutrient feed on day 6 and a 20 mM glucose feed on day 10 (“Initial feeding parameters”) or with two 0.7× concentration nutrient feeds on days 3 and 6, and three 30 mM glucose feeds on days 8, 10, and 12 (“Updated feeding parameters”). Depicted are titers after 14 days of total culture time, represented as grams per liter of culture.

FIG. 2 shows that an updated dulaglutide manufacturing scheme, according to aspects of the present disclosure, results in increased protease clipping. Mammalian cell cultures were fed as described for FIG. 1. Protease clipping of dulaglutide was evaluated at day 14 by measuring levels of the des H/HG variant. Depicted are the amounts of des H/HG for each condition as a percentage of total dulaglutide produced. The dashed line at 3.5% indicates the upper des H/HG % limit.

FIG. 3 shows that an optimized dulaglutide manufacturing scheme, according to aspects of the present disclosure, results in decreased protease clipping. Mammalian cell cultures were grown according to the conditions described in Example 2 and Table 3 herein, and protease clipping of dulaglutide was evaluated by measuring levels of the des H/HG variant. Depicted are the amounts of des H/HG for each condition as a percentage of total dulaglutide produced. The asterisks for Conditions 2 and 3 signify replicates. The dashed line at 3.5% indicates the upper des H/HG % limit.

### DETAILED DESCRIPTION

The present disclosure provides improved methods for producing Fc-containing proteins. The methods generally involve culturing a mammalian cell that expresses the Fc-containing protein in a production bioreactor for a first period of time followed by supplying a first nutrient feed and culturing the mammalian cell line for a second period of time followed by supplying a second nutrient feed. In some embodiments, the methods further involve culturing the mammalian cell line for a third period of time, followed by supplying a third nutrient feed. These methods are particularly advantageous in that they can promote efficient, high titer production of Fc-containing proteins while reducing protease clipping and unfavorable glycosylation of the Fc-containing proteins produced.

### I. DEFINITIONS

As used herein, the term “Fc-containing protein” refers to a protein comprising an Fc region. In an embodiment, the Fc-containing protein comprises a variant Fc region comprising one or more amino acid substitutions, additions, and/or deletions relative to a naturally occurring Fc region. In an embodiment, the Fc-containing protein is an antibody. In an embodiment, the Fc-containing protein is not an antibody.

As used herein, the term “antibody” includes full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above.

As used herein, the term “about,” when in reference to a value or parameter herein, includes a variability of +5% of the value or parameter. For example, when referring to an amount of protein having a particular characteristic (e.g., N-terminal clipping), “about” refers to a range that includes the value 5% below the referenced value, and the value 5% above the referenced value. Thus, a plurality of proteins with about 10% being an N-terminally clipped variant refers to a plurality of proteins that encompasses 9.5% to 10.5% of the proteins being an N-terminally clipped variant.

### II. METHODS OF CULTURING MAMMALIAN CELLS

When producing an Fc-containing protein, optimizing the nutrient feeding conditions of a mammalian cell culture in a production bioreactor is important to maintain the product quality of the Fc-containing protein. Disclosed herein are methods of producing an Fc-containing protein comprising culturing a mammalian cell that expresses the Fc-containing protein in a production bioreactor for a first period of time followed by supplying a first nutrient feed and culturing the mammalian cell line for a second period of time followed by supplying a second nutrient feed. In some embodiments, the methods further involve culturing the mammalian cell line for a third period of time, followed by supplying a third nutrient feed. The methods disclosed herein are particularly advantageous in that they can reduce protease clipping and unfavorable glycosylation of the Fc-containing protein in the mammalian cell culture, thereby increasing the amount of intact Fc-containing protein. In an embodiment, the Fc-containing protein is a dulaglutide protein, as described in further detail below.

In an aspect, the methods disclosed herein comprise culturing a mammalian cell that expresses an Fc-containing protein in a production bioreactor for a total culture time, wherein a) a first nutrient feed is supplied to the production bioreactor after a first portion of the total culture time; b) a second nutrient feed is supplied to the production bioreactor after a second portion of the total culture time; and c) a third nutrient feed is supplied to the production bioreactor after a third portion of the total culture time, such that the Fc-containing protein is produced by the mammalian cell.

In an aspect, the methods disclosed herein comprise culturing a mammalian cell that expresses a dulaglutide protein in a production bioreactor for a total culture time, wherein a) a first nutrient feed is supplied to the production bioreactor after a first portion of the total culture time; b) a second nutrient feed is supplied to the production bioreactor after a second portion of the total culture time; and c) a third nutrient feed is supplied to the production bioreactor after a third portion of the total culture time, such that the dulaglutide protein is produced by the mammalian cell.

Exemplary cell culture methods and conditions suitable for use in the foregoing methods are described in detail below.

The nutrient feed or nutrient feeds supplied to the cell cultures in the methods disclosed herein can comprise any nutrient feed composition known to the skilled artisan that is suitable for culturing mammalian cells. Generally, a nutrient feed composition will comprise a single nutrient feed component or a mixture of nutrient feed components. A nutrient feed may comprise supplying one or more nutrient feed components to the cell culture simultaneously (e.g., as a single mixture or as one or more separate mixtures supplied to the cell culture at the same time). In an embodiment of the methods provided herein, the first nutrient feed and second nutrient feed comprise the same nutrient feed composition. In an embodiment, the first nutrient feed and the second nutrient feed comprise different nutrient feed compositions. In an embodiment, the first nutrient feed, the second nutrient feed, and/or the third nutrient feed comprise the same nutrient feed compositions. In an embodiment, the first nutrient feed, the second nutrient feed, and/or the third nutrient feed comprise different nutrient feed compositions.

In an embodiment, nutrient feeds useful in the methods provided herein comprise nutrient feed compositions or nutrient feed components that have been used in previously described methods. For example, the nutrient feeds may comprise commercially available nutrient feed components or mixtures, or the nutrient feeds may comprise nutrient feed components or mixtures used in previously developed methods of producing Fc-containing proteins (e.g., dulaglutide).

In an embodiment of the methods provided herein, a nutrient feed comprises a nutrient feed component present at a reduced concentration relative to the concentration of said component in a previously described method. For example, where the nutrient feed component is commercially available, it may be used in the methods provided herein at a reduced concentration relative to the concentration recommended in the manufacturer protocol. As another example, where the nutrient feed component is used at a particular concentration in a previously developed method of producing an Fc-containing protein (e.g., dulaglutide), the nutrient feed component may be used in the methods provided herein at a reduced concentration relative to said particular concentration. In an embodiment of the methods provided herein, a nutrient feed comprises a nutrient feed component present at a concentration of about 0.05× to 0.95× (e.g., 0.1×, 0.15×, 0.2×, 0.25×, 0.3×, 0.35×, 0.4×, 0.45×, 0.5×, 0.55×, 0.6×, 0.65×, 0.7×, 0.75×, 0.8×, 0.85×, or 0.9×) relative to the concentration of the component in a previously described method.

In an embodiment of the methods provided herein, a nutrient feed comprises a nutrient feed component present at an increased concentration relative to the concentration of said component in a previously described method. In an embodiment, a nutrient feed comprises a nutrient feed component present at a concentration of about 1.05× to 10× (e.g., 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 2×, 2.1×, 2.15×, 2.2×, 2.25×, 2.3×, 2.35×, 2.4×, 2.45×, 2.5×, 2.55×, 2.6×, 2.65×, 2.7×, 2.75×, 2.8×, 2.85×, 2.9×, 3×, 3.1×, 3.15×, 3.2×, 3.25×, 3.3×, 3.35×, 3.4×, 3.45×, 3.5×, 3.55×, 3.6×, 3.65×, 3.7×, 3.75×, 3.8×, 3.85×, 3.9×, 4×, 4.1×, 4.15×, 4.2×, 4.25×, 4.3×, 4.35×, 4.4×, 4.45×, 4.5×, 4.55×, 4.6×, 4.65×, 4.7×, 4.75×, 4.8×, 4.85×, 4.9×, 5×, 5.25×, 5.5×, 5.75×, 6×, 6.25×, 6.5×, 6.75×, 7×, 7.25×, 7.5×, 7.75×, 8×, 8.25×, 8.5×, 8.75×, 9×, 9.25×, 9.5×, or 9.75×) relative to the concentration of the component in a previously described method.

In an embodiment, a nutrient feed comprises a nutrient feed component or components that are present at a defined weight relative to the total weight of the cell culture to which the nutrient feed is supplied. For example, a nutrient feed component may be present in the nutrient feed at about 1 gram of nutrient feed component per kilogram of cell culture (g/kg) to about 200 g/kg (e.g., about 1 g/kg, about 2 g/kg, about 4 g/kg, about 6 g/kg, about 8 g/kg, about 10 g/kg, about 12 g/kg, about 14 g/kg, about 16 g/kg, about 18 g/kg, about 20 g/kg, about 22 g/kg, about 24 g/kg, about 26 g/kg, about 28 g/kg, about 30 g/kg, about 32 g/kg, about 34 g/kg, about 36 g/kg, about 38 g/kg, about 40 g/kg, about 42 g/kg, about 44 g/kg, about 46 g/kg, about 48 g/kg, about 50 g/kg, about 52 g/kg, about 54 g/kg, about 56 g/kg, about 58 g/kg, about 60 g/kg, about 62 g/kg, about 64 g/kg, about 66 g/kg, about 68 g/kg, about 70 g/kg, about 72 g/kg, about 74 g/kg, about 76 g/kg, about 78 g/kg, about 80 g/kg, about 82 g/kg, about 84 g/kg, about 86 g/kg, about 88 g/kg, about 90 g/kg, about 92 g/kg, about 94 g/kg, about 96 g/kg, about 98 g/kg, about 100 g/kg, about 102 g/kg, about 104 g/kg, about 106 g/kg, about 108 g/kg, about 110 g/kg, about 112 g/kg, about 114 g/kg, about 116 g/kg, about 118 g/kg, about 120 g/kg, about 122 g/kg, about 124 g/kg, about 126 g/kg, about 128 g/kg, about 130 g/kg, about 132 g/kg, about 134 g/kg, about 136 g/kg, about 138 g/kg, about 140 g/kg, about 142 g/kg, about 144 g/kg, about 146 g/kg, about 148 g/kg, about 150 g/kg, about 152 g/kg, about 154 g/kg, about 156 g/kg, about 158 g/kg, about 160 g/kg, about 162 g/kg, about 164 g/kg, about 166 g/kg, about 168 g/kg, about 170 g/kg, about 172 g/kg, about 174 g/kg, about 176 g/kg, about 178 g/kg, about 180 g/kg, about 182 g/kg, about 184 g/kg, about 186 g/kg, about 188 g/kg, about 190 g/kg, about 192 g/kg, about 194 g/kg, about 196 g/kg, about 198 g/kg, or about 200 g/kg).

In an embodiment, the methods provided herein comprise supplying L-tyrosine to the mammalian cell culture. In an embodiment, one or more of the nutrient feeds comprise L-tyrosine as a nutrient feed component (i.e., the nutrient feed composition comprises L-tyrosine, or L-tyrosine is supplied to the cell culture separately but concurrently with the nutrient feed). In an embodiment, the first nutrient feed and/or the second nutrient feed comprises L-tyrosine. In an embodiment, the first nutrient feed and the second nutrient feed comprise L-tyrosine. In an embodiment, the first nutrient feed, the second nutrient feed, and/or the third nutrient feed comprises L-tyrosine. In an embodiment, the first nutrient feed, the second nutrient feed, and the third nutrient feed comprise L-tyrosine.

In an embodiment, the L-tyrosine is supplied to the mammalian cell culture as part of an L-tyrosine solution having a concentration of about 1% to about 10% (e.g., about 1%, about 1.2%, about 1.4%, about 1.6%, about 1.8%, about 2%, about 2.2%, about 2.4%, about 2.6%, about 2.8%, about 3%, about 3.2%, about 3.4%, about 3.6%, about 3.8%, about 4%, about 4.2%, about 4.4%, about 4.6%, about 4.8%, about 5%, about 5.2%, about 5.4%, about 5.6%, about 5.8%, about 6%, about 6.2%, about 6.4%, about 6.6%, about 6.8%, about 7%, about 7.2%, about 7.4%, about 7.6%, about 7.8%, about 8%, about 8.2%, about 8.4%, about 8.6%, about 8.8%, about 9%, about 9.2%, about 9.4%, about 9.6%, about 9.8%, or about 10%) L-tyrosine. In an embodiment, the L-tyrosine solution has a concentration of about 3.5%. In an embodiment, the L-tyrosine solution has a concentration of 3.5% or 35 g/L of L-tyrosine dissolved in water.

In an embodiment, the L-tyrosine solution is supplied to the mammalian cell culture at a concentration of about 1 g of 3.5% L-tyrosine solution/1 kg of bioreactor weight (i.e., 1 g/kg) to about 12 g/kg (e.g., about 1 g/kg, about 1.2 g/kg, about 1.4 g/kg, about 1.6 g/kg, about 1.8 g/kg, about 2 g/kg, about 2.2 g/kg, about 2.4 g/kg, about 2.6 g/kg, about 2.8 g/kg, about 3 g/kg, about 3.1 g/kg, about 3.2 g/kg, about 3.3 g/kg, about 3.4 g/kg, about 3.5 g/kg, about 3.6 g/kg, about 3.7 g/kg, about 3.8 g/kg, about 3.9 g/kg, about 4 g/kg, about 4.1 g/kg, about 4.2 g/kg, about 4.3 g/kg, about 4.4 g/kg, about 4.5 g/kg, about 4.6 g/kg, about 4.7 g/kg, about 4.8 g/kg, about 4.9 g/kg, about 5 g/kg, about 5.1 g/kg, about 5.2 g/kg, about 5.3 g/kg, about 5.4 g/kg, about 5.5 g/kg, about 5.6 g/kg, about 5.7 g/kg, about 5.8 g/kg, about 5.9 g/kg, about 6 g/kg, about 6.1 g/kg, about 6.2 g/kg, about 6.3 g/kg, about 6.4 g/kg, about 6.5 g/kg, about 6.6 g/kg, about 6.7 g/kg, about 6.8 g/kg, about 6.9 g/kg, about 7 g/kg, about 7.1 g/kg, about 7.2 g/kg, about 7.3 g/kg, about 7.4 g/kg, about 7.5 g/kg, about 7.6 g/kg, about 7.7 g/kg, about 7.8 g/kg, about 7.9 g/kg, about 8 g/kg, about 8.1 g/kg, about 8.2 g/kg, about 8.3 g/kg, about 8.4 g/kg, about 8.5 g/kg, about 8.6 g/kg, about 8.7 g/kg, about 8.8 g/kg, about 8.9 g/kg, about 9 g/kg, about 9.2 g/kg, about 9.4 g/kg, about 9.6 g/kg, about 9.8 g/kg, about 10 g/kg, about 10.2 g/kg, about 10.4 g/kg, about 10.6 g/kg, about 10.8 g/kg, about 11 g/kg, about 11.2 g/kg, about 11.4 g/kg, about 11.6 g/kg, about 11.8 g/kg, or about 12 g/kg). In an embodiment, the L-tyrosine solution is supplied to the mammalian cell culture at a concentration of about 4.1 g/kg. In an embodiment, the L-tyrosine solution is supplied to the mammalian cell culture at a concentration of 4.1 g/kg. In an embodiment, the L-tyrosine solution is supplied to the mammalian cell culture at a concentration of about 7.7 g/kg. In an embodiment, the L-tyrosine solution is supplied to the mammalian cell culture at a concentration of 7.7 g/kg.

In an embodiment, the methods provided herein comprise supplying glucose to the mammalian cell culture. In an embodiment, the glucose level of the cell culture in the bioreactor is monitored, and glucose is supplied when the glucose level is below a threshold. In an embodiment, glucose is supplied when the glucose level is below about 20 mM (e.g., below about 19 mM, below about 18 mM, below about 17 mM, below about 16 mM, below about 15 mM, below about 14 mM, below about 13 mM, below about 12 mM, below about 11 mM, or below about 10 mM). In an embodiment, glucose is supplied when the glucose level is below about 15 mM. In an embodiment, glucose is supplied when the glucose level is below 15 mM.

In an embodiment, glucose is supplied to the mammalian cell culture on set days during the total culture time. In an embodiment, glucose is supplied on any of days 5, 6, 7, 8, 9, 10, 11, 12, 13, 14, 15, 16, 17, 18, 19, and/or 20. In an embodiment, glucose is supplied once during the total culture time. In an embodiment, glucose is supplied more than once (e.g., two times, three times, four times, five times, or more than five times) during the total culture time. In an embodiment, glucose is supplied on day 10. In an embodiment, glucose is supplied on days 8 and 11, days 8 and 13, days 11 and 13, or days 8, 11, and 13. In an embodiment, glucose is supplied on days 8 and 10, days 8 and 12, days 10 and 12, or days 8, 10, and 12.

In an embodiment, glucose is supplied to the mammalian cell culture at a concentration of about 10 mM, about 12 mM, about 14 mM, about 16 mM, about 18 mM, about 20 mM, about 22 mM, about 24 mM, about 26 mM, about 28 mM, about 30 mM, about 32 mM, about 34 mM, about 36 mM, about 38 mM, or about 40 mM. In an embodiment, glucose is supplied at about 20 mM. In an embodiment, glucose is supplied at 20 mM. In an embodiment, glucose is supplied at about 30 mM. In an embodiment, glucose is supplied at 30 mM. In an embodiment, glucose is supplied at 20 mM on day 10. In an embodiment, glucose is supplied at 20 mM on day 8 and 30 mM on day 11, at 20 mM on day 8 and 30 mM on day 13, at 30 mM on day 11 and 30 mM on day 13, or at 20 mM on day 8, 30 mM on day 11, and 30 mM on day 13. In an embodiment, glucose is supplied at 30 mM on days 8 and 10, at 30 mM on days 8 and 12, at 30 mM on days 10 and 12, or at 30 mM on days 8, 10, and 12.

In an embodiment of the methods provided herein, the total culture time is at least 8 days (e.g., at least 9 days, at least 10 days, at least 11 days, at least 12 days, at least 13 days, at least 14 days, at least 15 days, at least 16 days, at least 17 days, at least 18 days, at least 19 days, or at least 20 days).

In an embodiment, the total culture time is less than 20 days (e.g., less than 19 days, less than 18 days, less than 17 days, less than 16 days, less than 15 days, less than 14 days, less than 13 days, less than 12 days, less than 11 days, less than 10 days, less than 9 days, or less than 8 days).

In an embodiment, the total culture time is from 8-20 days (e.g., 9-19 days, 10-18 days, 11-17 days, 12-16 days, 13-15 days, 8-12 days, 8-16 days, 10-20 days, 10-16 days, 10-14 days, 12-20 days, 12-18 days, 12-14 days, 14-20 days, 14-18 days, 14-16 days, 16-20 days, 16-18 days, or 18-20 days). In an embodiment, the total culture time is about 12 to about 16 days. In an embodiment, the total culture time is 12-16 days. In an embodiment, the total culture time is about 8 days, about 9 days, about 10 days, about 11 days, about 12 days, about 13 days, about 14 days, about 15 days, about 16 days, about 17 days, about 18 days, about 19 days, or about 20 days. In an embodiment, the total culture time is 8 days, 9 days, 10 days, 11 days, 12 days, 13 days, 14 days, 15 days, 16 days, 17 days, 18 days, 19 days, or 20 days. In an embodiment, the total culture time is about 14 days. In an embodiment, the total culture time is 14 days.

In an embodiment of the methods provided herein, the first portion of the total culture time is about 10% to about 40% (e.g., about 15% to about 35%, about 20% to about 30%, about 10% to about 30%, about 10% to about 20%, about 15% to about 30%, about 20% to about 35%, about 20% to about 40%, about 25% to about 40%, or about 30% to about 40%) of the total culture time. In an embodiment of the methods provided herein, the first portion of the total culture time is about 20% to about 30% of the total culture time. In an embodiment of the methods provided herein, the first portion of the total culture time is 20% to 30% of the total culture time. In an embodiment, the first portion of the total culture time is from 1-5 days (e.g., 1-4 days, 1-3 days, 1-2 days, 2-5 days, 2-4 days, 2-3 days, 3-5 days, 3-4 days, or 4-5 days). In an embodiment, the first portion of the total culture time is 2-4 days. In an embodiment, the first portion of the total culture time is about 1 day, about 2 days, about 3 days, about 4 days, or about 5 days. In an embodiment, the first portion of the total culture time is 1 day, 2 days, 3 days, 4 days, or 5 days. In an embodiment, the first portion of the total culture time is about 3 days. In an embodiment, the first portion of the total culture time is 3 days.

In an embodiment of the methods provided herein, the second portion of the total culture time is about 30% to about 60% (e.g., about 35% to about 55%, about 40% to about 50%, about 30% to about 50%, about 30% to about 40%, about 35% to about 50%, about 40% to about 55%, about 40% to about 60%, about 45% to about 60%, or about 50% to about 60%) of the total culture time. In an embodiment of the methods provided herein, the second portion of the total culture time is about 40% to about 50% of the total culture time. In an embodiment of the methods provided herein, the second portion of the total culture time is 40% to 50% of the total culture time. In an embodiment, the second portion of the total culture time is from 4-8 days (e.g., 4-7 days, 4-6 days, 4-5 days, 5-8 days, 5-7 days, 5-6 days, 6-8 days, 6-7 days, or 7-8 days). In an embodiment, the second portion of the total culture time is 5-7 days. In an embodiment, the second portion of the total culture time is about 4 days, about 5 days, about 6 days, about 7 days, or about 8 days. In an embodiment, the second portion of the total culture time is 4 days, 5 days, 6 days, 7 days, or 8 days. In an embodiment, the second portion of the total culture time is about 6 days. In an embodiment, the second portion of the total culture time is 6 days.

In an embodiment of the methods provided herein, the third portion of the total culture time is about 50% to about 80% (e.g., about 55% to about 75%, about 60% to about 70%, about 50% to about 70%, about 50% to about 60%, about 55% to about 70%, about 60% to about 75%, about 60% to about 80%, about 65% to about 80%, or about 70% to about 80%) of the total culture time. In an embodiment of the methods provided herein, the third portion of the total culture time is about 60% to about 70% of the total culture time. In an embodiment of the methods provided herein, the third portion of the total culture time is 60% to 70% of the total culture time. In an embodiment, the third portion of the total culture time is from 7-11 days (e.g., 7-10 days, 7-9 days, 7-8 days, 8-11 days, 8-10 days, 8-9 days, 9-11 days, 9-10 days, or 10-11 days). In an embodiment, the third portion of the total culture time is 8-10 days. In an embodiment, the third portion of the total culture time is about 7 days, about 8 days, about 9 days, about 10 days, or about 11 days. In an embodiment, the third portion of the total culture time is 7 days, 8 days, 9 days, 10 days, or 11 days. In an embodiment, the third portion of the total culture time is about 9 days. In an embodiment, the third portion of the total culture time is 9 days.

In an embodiment, the first portion of the total culture time is about 20% to about 30% of the total culture time and the second portion of the total culture time is about 40% to about 50% of the total culture time. In an embodiment, the first portion of the total culture time is 20% to 30% of the total culture time and the second portion of the total culture time is 40% to 50% of the total culture time. In an embodiment, the first portion of the total culture time is 2-4 days and the second portion of the total culture time is 5-7 days. In an embodiment, the first portion of the total culture time is 3 days and the second portion of the total culture time is 6 days.

In an embodiment, the first portion of the total culture time is about 20% to about 30% of the total culture time, the second portion of the total culture time is about 40% to about 50% of the total culture time, and the third portion of the total culture time is about 60% to about 70% of the total culture time. In an embodiment, the first portion of the total culture time is 20% to 30% of the total culture time, the second portion of the total culture time is 40% to 50% of the total culture time, and the third portion of the total culture time is 60% to 70% of the total culture time. In an embodiment, the first portion of the total culture time is 2-4 days, the second portion of the total culture time is 5-7 days, and the third portion of the total culture time is 8-10 days. In an embodiment, the first portion of the total culture time is 3 days, the second portion of the total culture time is 6 days, and the third portion of the total culture time is 9 days.

In an embodiment, the methods provided herein comprise culturing the mammalian cell at a first temperature for 1-5 days followed by culturing the mammalian cell at a second temperature for 9-14 days. In an embodiment, the method further comprises culturing the mammalian cell at a first temperature for about 1 day followed by culturing the mammalian cell at a second temperature for about 9, about 10, about 11, about 12, about 13, or about 14 days. In an embodiment, the method further comprises culturing the mammalian cell at a first temperature for about 2 days followed by culturing the mammalian cell at a second temperature for about 9, about 10, about 11, about 12, about 13, or about 14 days. In an embodiment, the method further comprises culturing the mammalian cell at a first temperature for about 3 days followed by culturing the mammalian cell at a second temperature for about 9, about 10, about 11, about 12, about 13, or about 14 days. In an embodiment, the method further comprises culturing the mammalian cell at a first temperature for about 4 days followed by culturing the mammalian cell at a second temperature for about 9, about 10, about 11, about 12, about 13, or about 14 days. In an embodiment, the method further comprises culturing the mammalian cell at a first temperature for about 5 days followed by culturing the mammalian cell at a second temperature for about 9, about 10, about 11, about 12, about 13, or about 14 days.

In an embodiment, the second temperature is lower than the first temperature. In an embodiment, the second temperature is about 1 °C, about 2 °C, about 3 °C, about 4 °C, about 5 °C, or about 6 °C lower than the first temperature.

In an embodiment, the first temperature is 34 °C-40 °C. In an embodiment, the first temperature is about 34 °C, about 35 °C, about 36 °C, about 37 °C, about 38 °C, about 39 °C, or about 40 °C. In an embodiment, the first temperature is about 36 °C.

In an embodiment, the second temperature is 30 °C-36 °C. In an embodiment, the second temperature is about 30 °C, about 31 °C, about 32 °C, about 33 °C, about 34 °C, about 35 °C, or about 36 °C. In an embodiment, the second temperature is about 33 °C.

In an embodiment, the mammalian cell is cultured at the first temperature for 3-4 days. In an embodiment, the mammalian cell is cultured at the first temperature for about 3 days. In an embodiment, the mammalian cell is cultured at the second temperature for 11-12 days. In an embodiment, the mammalian cell is cultured at the second temperature for about 11 days.

In an embodiment of the methods provided herein, the mammalian cell is cultured in a bioreactor. In an embodiment, the mammalian cell is cultured in fed batch mode. Examples of bioreactors include, but are not limited to, a plug flow bioreactor, a continuous stirred tank bioreactor, a fixed-bed bioreactor, an air-lift bioreactor, and a bioreactor bag. In an embodiment, the maximum viable cell density (VCD) in the production bioreactor is between about 4×10⁶ and about 18×10⁶ (e.g., between about 6×10⁶ and about 16×10⁶, between about 8×10⁶ and about 14×10⁶, between about 10×10⁶ and about 12×10⁶, between about 4×10⁶ and about 14×10⁶, between about 4×10⁶ and about 10×10⁶, between about 6×10⁶ and about 18×10⁶, between about 6×10⁶ and about 14×10⁶, between about 6×10⁶ and about 10×10⁶, between about 8×10⁶ and about 18×10⁶, between about 8×10⁶ and about 14×10⁶, between about 8×10⁶ and about 10×10⁶, between about 10×10⁶ and about 18×10⁶, between about 10×10⁶ and about 14×10⁶, between about 12×10⁶ and about 18×10⁶, between about 12×10⁶ and about 14×10⁶, or between about 14×10⁶ and about 18×10⁶) cells per milliliter (cells/mL). In an embodiment, the maximum VCD in the production bioreactor is between about 8×10⁶ and about 14×10⁶ cells/mL. In an embodiment, the maximum VCD in the production bioreactor is between 8×10⁶ and 14×10⁶ cells/mL.

In some embodiment, the mammalian cell is selected from the group consisting of a COS cell, a CHO cell, a BHK cell, an MDCK cell, a HEK293 cell, a HEK293T cell, a HeLa cell, an NS0 cell, a PER.C6 cell, a VERO cell, a CRL7030 cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. In an embodiment, the mammalian cell is a CHO cell.

The cell culture medium used in the methods disclosed herein can comprise any medium known to the skilled artisan that is suitable for culturing mammalian cells.

In an embodiment, less than about 4% of the Fc-containing protein produced is N-terminally clipped. In an embodiment, less than about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2.0%, about 1.9%, about 1.8%, about 1.7%, about 1.6%, about 1.5%, about 1.4%, about 1.3%, about 1.2%, about 1.1%, or about 1.0% of the Fc-containing protein produced is N-terminally clipped.

In an embodiment, less than about 4% of the dulaglutide produced is N-terminally clipped. In an embodiment, less than about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, or about 2.5% of the dulaglutide produced is N-terminally clipped. In an embodiment, the N-terminally clipped dulaglutide does not have the N-terminal H1 or G2 residues of dulaglutide.

In an embodiment, less than about 5% (e.g., less than about 4.5%, less than about 4%, less than about 3.5%, less than about 3%, less than about 2.5%, less than about 2%, less than about 1.5%, less than about 1%, or less than about 0.5%) of the Fc-containing protein (e.g., dulaglutide) produced comprises a G2F glycan. In an embodiment, between about 0.5% and about 4% (e.g., between about 1% and about 3.5%, between about 1.5% and about 3%, between about 1.5% and about 2.6%, or between about 2% and about 2.5%) of the Fc-containing protein (e.g., dulaglutide) produced comprises a G2F glycan. In an embodiment, between about 1.5% and about 2.6% of the Fc-containing protein (e.g., dulaglutide) produced comprises a G2F glycan. In an embodiment, between 1.5% and 2.6% of the Fc-containing protein (e.g., dulaglutide) produced comprises a G2F glycan.

In an embodiment, less than about 20% (e.g., less than about 19.5%, less than about 19%, less than about 18.5%, less than about 18%, less than about 17.5%, less than about 17%, less than about 16.5%, less than about 16%, less than about 15.5%, less than about 15%, less than about 14.5%, less than about 14%, less than about 13.5%, less than about 13%, less than about 12.5%, less than about 12%, less than about 11.5%, less than about 11%, less than about 10.5%, or less than about 10%) of the Fc-containing protein (e.g., dulaglutide) produced comprises a GIF glycan. In an embodiment, between about 8% and about 18% (e.g., between about 9% and about 17%, between about 10% and about 16%, between about 10.6% and about 15.4%, between about 11% and about 15%, or between about 12% and about 14%) of the Fc-containing protein (e.g., dulaglutide) produced comprises a GIF glycan. In an embodiment, between about 10.6% and about 15.4% of the Fc-containing protein (e.g., dulaglutide) produced comprises a GIF glycan. In an embodiment, between 10.6% and 15.4% of the Fc-containing protein (e.g., dulaglutide) produced comprises a GIF glycan.

In an embodiment, less than about 90% (e.g., less than about 88%, less than about 86%, less than about 84%, less than about 82%, less than about 80%, less than about 78%, less than about 76%, less than about 74%, or less than about 72%) of the Fc-containing protein (e.g., dulaglutide) produced comprises a GOF glycan. In an embodiment, between about 70% and about 85% (e.g., between about 71% and about 84%, between about 72% and about 83%, between about 73% and about 82%, between about 74% and about 81%, between about 74.7% and about 80%, between about 75% and about 80%, or between about 74% and about 79%) of the Fc-containing protein (e.g., dulaglutide) produced comprises a GOF glycan. In an embodiment, between about 74.7% and about 80% of the Fc-containing protein (e.g., dulaglutide) produced comprises a GOF glycan. In an embodiment, between 74.7% and 80% of the Fc-containing protein (e.g., dulaglutide) produced comprises a GOF glycan.

In an embodiment, less than about 4% (e.g., less than about 4%, less than about 3.8%, less than about 3.6%, less than about 3.4%, less than about 3.2%, less than about 3%, less than about 2.8%, less than about 2.6%, less than about 2.4%, less than about 2.2%, less than about 2%, less than about 1.8%, less than about 1.6%, less than about 1.4%, less than about 1.2%, less than about 1%, less than about 0.8%, less than about 0.6%, less than about 0.4%, or less than about 0.2%) of the Fc-containing protein (e.g., dulaglutide) produced comprises a Man-5 glycan. In an embodiment, less than about 1% of the Fc-containing protein (e.g., dulaglutide) produced comprises a Man-5 glycan. In an embodiment, less than 1% of the Fc-containing protein (e.g., dulaglutide) produced comprises a Man-5 glycan.

### III. FC-CONTAINING PROTEINS

The methods provided by the present disclosure are for the production of an Fc-containing protein by culturing a mammalian cell that expresses the Fc-containing protein.

In an embodiment, the Fc-containing protein comprises one or more of the amino acid sequences set forth in Table 1 below.

In an embodiment, the Fc-containing protein comprises a glucagon-like peptide 1 (GLP-1) analog comprising one or more modifications compared to a wild type GLP-1 amino acid sequence (SEQ ID NO: 1).

In an embodiment, the Fc-containing protein comprises a GLP-1 analog comprising the amino acid sequence of SEQ ID NO: 2.

In an embodiment, the Fc-containing protein comprises a peptide linker. In an embodiment, the C-terminal amino acid of the GLP-1 analog portion of the Fc-containing protein is fused to the N-terminus of an Fc portion of an immunoglobulin via a peptide linker. In an embodiment, the peptide linker comprises 1 to 10 G4S units (SEQ ID NO: 3).

In an embodiment, the Fc-containing protein comprises: a GLP-1 analog comprising the amino acid sequence of SEQ ID NO: 2; a peptide linker comprising 1 to 10 G4S units (SEQ ID NO: 3); and an Fc portion of an immunoglobulin. In an embodiment, the N-terminal residue of the peptide linker is directly fused to the C-terminal residue of the GLP-1 analog, and the C-terminal residue of the peptide linker is directly fused to the N-terminal residue of the Fc portion.

In an embodiment, the Fc-containing protein comprises the amino acid sequence of SEQ ID NO: 4. In an embodiment, the wherein the Fc-containing protein is a homodimer comprising two identical amino acid chains each comprising the amino acid sequence of SEQ ID NO: 4.

In an embodiment, the Fc-containing protein is dulaglutide.

Dulaglutide is a human GLP-1 receptor agonist which comprises a dimer of a GLP-1 analog fused at its C-terminus via a (G4S) 3 peptide linker to the N-terminus of an analog of an Fc portion of an immunoglobulin, and is identified by CAS registry number 923950-08-7, which provides the following chemical name: 7-37-Glucagon-like peptide I [8-glycine, 22-glutamic acid, 36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO: 4.

The two monomers are attached by disulfide bonds between the cysteine residues at positions 55 and 58 of SEQ ID NO: 4 to form the dimer. Dulaglutide's structure, function, production, and use in treating T2DM is described in more detail in U.S. Pat. No. 7,452,966 and U.S. Patent Application Publication No. US20100196405. Dulaglutide agonizes the GLP-1 receptor resulting in stimulation of insulin synthesis and secretion and has been shown to provide improved glycemic control in T2DM patients.

When used herein, the term “dulaglutide” refers to any GLP-1 receptor agonist protein dimer of two monomers having the amino acid sequence of SEQ ID NO: 4, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term.

TABLE 1
Sequences comprised in certain Fc-containing proteins.

| | SEQ ID | |
|---|---|---|
| Description | NO: | Amino acid sequence |
| WT GLP 1 | 1 | HAEGTFTSDVSSYLEGQAAKEFIAWLVKGRG |
| GLP 1 | 2 | HGEGTFTSDVSSYLEEQAAKEFIAWLVKGGG |
| analog | | |
| G₄S | 3 | GGGGS |
| Dulaglutide | 4 | HGEGTFTSDVSSYLEEQAAKEFIAWLVKGGGGGGGSGGGGSGGGGSAE |
| monomer | | SKYGPPCPPCPAPEAAGGPSVFLFPPKPKDTLMISRTPEVTCVVVDVSQE |
| | | DPEVQFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQVSLT |
| | | CLVKGFYPSDIAVEWESNGQPENNYKTTPPVLDSDGSFFLYSRLTVDKS |
| | | RWQEGNVFSCSVMHEALHNHYTQKSLSLSLG |

In an embodiment, the Fc-containing protein is etanercept, alefacept, abatacept, rilonacept, romiplostim, belatacept, aflibercept, conbercept, efmoroctocog alpha, eftrenonacog alpha, asfotase alpha, or luspatercept.

In an embodiment, the Fc-containing protein is an antibody. In an embodiment, the Fc-containing protein is not an antibody.

In an aspect, provided herein is an Fc-containing protein produced by any one of the methods disclosed herein.

In an aspect, provided herein is dulaglutide produced by any one of the methods disclosed herein.

### EXAMPLES

The following examples are offered by way of illustration, and not by way of limitation.

### Example 1: Optimization of Initial Dulaglutide Manufacturing Scheme

This Example describes studies that evaluated changes in feeding parameters during the production bioreactor stage of a dulaglutide manufacturing scheme. In an initial dulaglutide manufacturing scheme, the feeding parameters used in the production bioreactor stage-referred to herein as the “initial feeding parameters”—included a 1.5× concentration nutrient feed on day 6 and a 20 mM glucose feed on day 10. That initial dulaglutide manufacturing scheme was optimized to identify upstream conditions and feeding parameters that increase viable cell density (VCD) and maintain optimal product quality (e.g., minimal protease clipping, favorable glycosylation profiles, etc.). When upstream conditions were optimized to increase VCD, use of the initial feeding parameters in the production bioreactor resulted in nutrient depletion prior to protein harvest and thus decreased dulaglutide titer.

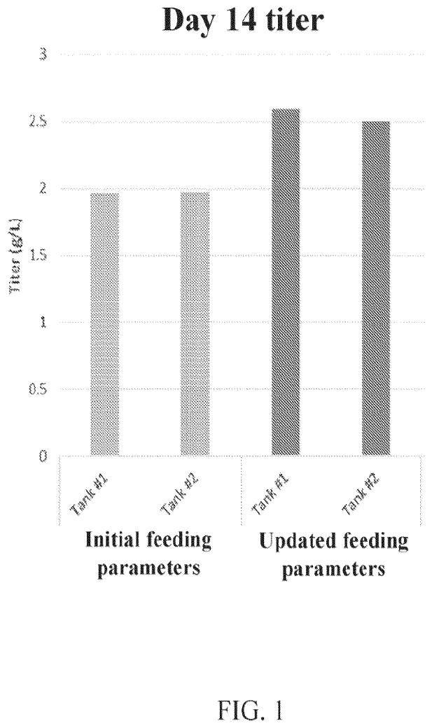

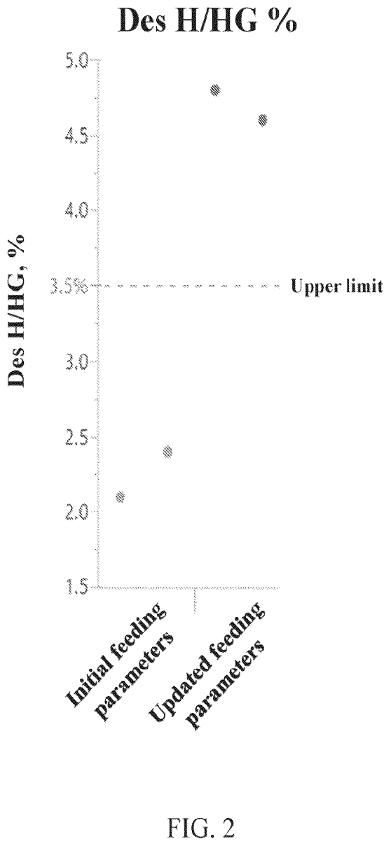

Several subsequent studies assessed the impact of changing feeding parameters used in the production bioreactor stage, leading to an updated dulaglutide manufacturing scheme including 2 nutrient feeds (0.7× concentration nutrient feeds on days 3 and 6) and 3 glucose feeds (30 mM glucose feeds on days 8, 10, and 12). The updated dulaglutide manufacturing scheme feeding parameters are referred to in this Example as “updated feeding parameters.” These studies demonstrated an increase in VCD and dulaglutide titer (FIG. 1) over the initial dulaglutide manufacturing scheme, but also demonstrated increased protease clipping of dulaglutide (e.g., clipping of the H1 residue or both of the H1 and G2 residues from the N-terminus, resulting in the des H/HG variant of dulaglutide; FIG. 2) and an undesirable shift in glycoform distribution, as shown in Table 2. Based on this, it was suspected that certain nutrients may be limiting towards the end of the culture period in the updated dulaglutide manufacturing scheme. Further analysis indicated that the level of L-tyrosine was exhausted by Day 14. In this context of this Example and Example 2 below, references to “X” with respect to L-tyrosine refer to addition of 5 g of 3.5% L-tyrosine solution per kg of mass in the bioreactor, or “5 g/kg.” Thus, for example, addition of 1.5× of a nutrient feed comprising L-tyrosine refers to addition of 7.5 g/kg, and 0.7× refers to addition of 3.5 g/kg.

TABLE 2
Quantification of dulaglutide glycoforms.

| | Initial | Initial | Updated | Updated |
|---|---|---|---|---|
| | feeding | feeding | feeding | feeding |
| | parameters | parameters | parameters | parameters |
| Glycoform | (200 L study) | (5 L study) | (200 L study) | (5 L study) |
| G2F % (1.5%-2.6%) | 2.4 | 2.1 | 3.3 | 3.1 |
| G1F isomers % | 14.7 | 13.8 | 17.6 | 17.2 |
| (10.6%-15.4%) | | | | |
| G0F % (74.7%-80%) | 75.2 | 76.2 | 69.1 | 69.8 |
| Man-5% (0%-1%) | 0.7 | 0.6 | 0.9 | 0.8 |
| G0 | 2 | 2 | 3.8 | 4 |
| Other % (5.7%-8.0%) | 5 | 5.3 | 5.3 | 5.1 |

### Example 2: Additional Optimization of Feeding Parameters to Maximize Product Quality

As described in Example 1, optimization of an initial dulaglutide manufacturing scheme resulted in increased protease clipping and an undesirable shift in glycoform distribution. This Example describes a study that evaluated further changes in the feeding parameters of the updated dulaglutide manufacturing scheme. Specifically, the effects of higher L-tyrosine feeds and the addition of a third nutrient feed during the production bioreactor stage on VCD, dulaglutide titer, and product quality (e.g., level of protease clipping, glycosylation profiles, etc.) were evaluated. A separate study was performed and showed that adjusting feed concentration from 0.7× to 0.8× resulted in more optimal glucose profiles while not significantly impacting titer. As such, the control nutrient and tyrosine feeds were adjusted from 0.7× (e.g., as described in Example 1) to 0.8×. As noted above, references to “X” with respect to L-tyrosine refer to addition of 5 g of 3.5% L-tyrosine solution per kg of mass in the bioreactor, so addition of a 0.7× L-tyrosine feed refers to addition of 3.5 g/kg and addition of a 0.8× L-tyrosine feed refers to addition of 4 g/kg.

For all conditions, glucose feeds were performed at 30 mM when glucose was measured at less than 15 mM. The feeding parameters tested are shown in Table 3, below.

TABLE 3
Feeding parameter conditions tested.

| | | Nutrient feed | Tyrosine feed | Nutrient feed |
|---|---|---|---|---|
| Condition | Description | concentration | concentration | days |
| 1 | Control | 0.8X | 0.8X | 3 and 6 |
| | (n = 1) | | | |
| 2 | Additional | 0.8X | 1X | 3 and 6 |
| | tyrosine | | | |
| | (n = 2) | | | |
| 3 | Additional | 0.8X | 0.8X | 3, 6, and 9 |
| | nutrient feed | | | |
| | (n = 2) | | | |

Similar growth and viability were observed across all 3 conditions, indicating that the additional nutrient feed and higher tyrosine concentration did not impact growth. Similarly, no significant differences in titer were observed across all 3 conditions.

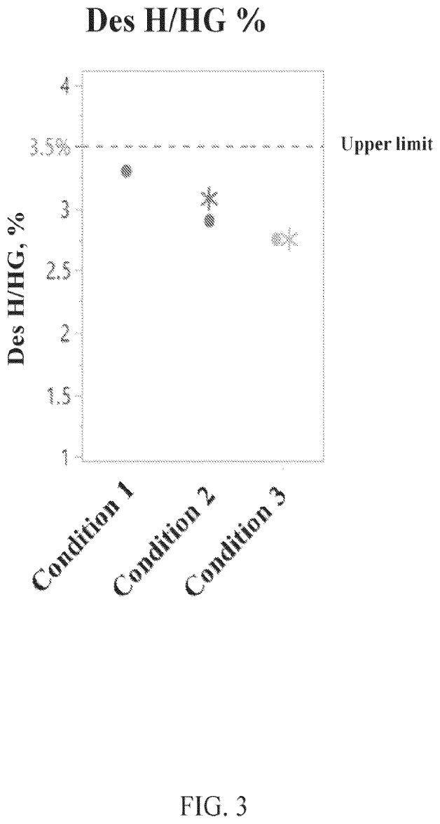

Product quality was measured across the 3 conditions using a variety of assays. Protease clipping of dulaglutide was evaluated by measuring the levels of des H/HG (FIG. 3). Compared to the control condition, des H/HG levels were decreased by approximately 10% and 17% with additional tyrosine feed and an additional nutrient feed, respectively. Measurement of glycoform levels, shown in Table 4, indicated that the additional nutrient feed condition resulted in acceptable levels for all measured glycoforms.

TABLE 4
Quantification of dulaglutide glycoforms.

| | | Additional | Additional |
|---|---|---|---|
| Glycoform (Limits) | Control | tyrosine | nutrient feed |
| G2F % (1.5%-2.6%) | 2.3 | 2.3 | 2.1 |
| G1F isomers % | 13.5 | 12.9 | 12.1 |
| (10.6%-15.4%) | | | |
| G0F % (74.7%-80%) | 73.9 | 73 | 75.4 |
| Man-5% (0%-1%) | 0.9 | 0.9 | 0.9 |
| G0 | 3.2 | 3.7 | 2.9 |
| Other % (5.7%-8.0%) | 6.1 | 7.2 | 6.8 |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Other embodiments are within the following claims.

1. A method for producing dulaglutide, the method comprising culturing a mammalian cell that expresses the dulaglutide in a production bioreactor for a total culture time, wherein:
a) a first nutrient feed is supplied to the production bioreactor after a first portion of the total culture time;
b) a second nutrient feed is supplied to the production bioreactor after a second portion of the total culture time; and
c) a third nutrient feed is supplied to the production bioreactor after a third portion of the total culture time,
wherein the first nutrient feed, the second nutrient feed, and/or the third nutrient feed comprises L-tyrosine,
such that the dulaglutide is produced by the mammalian cell.

2. (canceled)

3. The method of claim 1, wherein each of the first nutrient feed, the second nutrient feed, and the third nutrient feed comprises L-tyrosine.

4. The method of claim 1, wherein the L-tyrosine is supplied as a 3.5% solution.

5. The method of claim 1, wherein the L-tyrosine is supplied to the production bioreactor at a concentration of about 4 g/kg.

6-7. (canceled)

8. The method of claim 1, wherein the first portion of the total culture time is 2-4 days after initiation of said culturing.

9. The method of claim 1, wherein the first nutrient feed is supplied about 3 days after initiation of said culturing.

10. The method of claim 1, wherein the second portion of the total culture time is 5-7 days after initiation of said culturing.

11. The method of claim 1, wherein the second nutrient feed is supplied about 6 days after initiation of said culturing.

12. The method of claim 1, wherein the third portion of the total culture time is 8-10 days after initiation of said culturing.

13. The method of claim 1, wherein the third nutrient feed is supplied about 9 days after initiation of said culturing.

14. The method of claim 1, wherein the first nutrient feed is supplied about 3 days after initiation of said culturing, the second nutrient feed is supplied about 6 days after initiation of said culturing, and the third nutrient feed is supplied about 9 days after initiation of said culturing.

15. The method of claim 1, wherein the maximum viable cell density (VCD) in the production bioreactor is between 8×10⁶ and 14×10⁶ cells per milliliter.

16. The method of claim 1, wherein the mammalian cell is selected from the group consisting of a COS cell, a CHO cell, a BHK cell, an MDCK cell, a HEK293 cell, a HEK293T cell, a HeLa cell, an NS0 cell, a PER.C6 cell, a VERO cell, a CRL7030 cell, an HsS78Bst cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell.

17. The method of claim 1, wherein the mammalian cell is a CHO cell.

18. The method of claim 1, wherein less than about 4% of the dulaglutide produced is an N-terminally clipped variant.

19. The method of claim 18, wherein the N-terminally clipped variant is missing an N-terminal histidine residue or an N-terminal histidine residue and glycine residue.

20. The method of claim 1, wherein between 1.5% and 2.6% of the dulaglutide produced comprises a G2F glycan.

21. The method of claim 1, wherein between 10.6% and 15.4% of the dulaglutide produced comprises a GIF glycan or wherein between 74.7% and 80% of the dulaglutide produced comprises a GOF glycan.

22. (canceled)

23. The method of claim 1, wherein less than 1% of the dulaglutide produced comprises a Man-5 glycan.

24-30. (canceled)

31. A composition comprising dulaglutide and one or more N-terminally clipped variants of dulaglutide, wherein the composition comprises less than about 4% of the N-terminally clipped variant.
